(19) United States
(12) Patent Application Publication
(10) Pub. No.: US 2003/0157147 A1
(43) Pub. Date: Aug. 21, 2003

(54) Anti-microbial utility and kitchen wipe utilizing metallic silver as an oligodynamic agent

(76) Inventors: William Hoge, Palmyra, NY (US); Gary Winch, Naples, NY (US); David Yearsley, Export, PA (US)

Correspondence Address: Stephen B. Salai, Esq., Harter, Secrest & Emery LLP, 1600 Bausch & Lomb Place, Rochester, NY, 14604-2711, US

(21) Appl. No.: 10/077,317

(22) Filed: Feb. 15, 2002

(51) Int. Cl. A61K 9/70; B32B 27/04; B32B 27/12

(52) U.S. Cl. 424/443; 442/123

(57) ABSTRACT

A long-lasting antimicrobial wipe using metallic silver as the oligodynamic agent. The wipe has an outer layer of a flexible textile fabric having metallic silver deposited thereon, bonded to an inner layer of a fibrous water-retaining material. The silver is preferably deposited on the textile fabric by electroless coating, to provide efficient coverage of all available surfaces.

### FIELD OF THE INVENTION

This invention relates to an antimicrobial wipe, and in particular to a long-lasting wipe using metallic silver as an oligodynamic agent.

### BACKGROUND OF THE INVENTION

The need to maintain hygienic conditions in environments such as food preparation is well known. Precautions include washing hands and cleaning critical surfaces with suitable wipes.

Typically anti-bacterial chemicals are impregnated in these wipes to control anti-microbial contamination in food preparation and beauty salon work areas where such potentially dangerous microorganisms can be spread causing mass infections. These chemicals can cause resistant strains to develop rendering the wipes useless in the management of these infectious microorganisms. Excessive development of resistant microbes through the use of such chemicals, whether in wipes or otherwise, could potentially pose a public health hazard. Furthermore, wipes containing anti-microbial chemical agents are usually only good for single use and are sometimes toxic to small children. An uninformed user may habitually use a wipe on multiple occasions, unaware that it is no longer effective, and this itself may create a hazardous situation.

Therefore, there is a need for an inexpensive antimicrobial wipe that retains its antimicrobial activity through repeated uses. There is further a need for an antimicrobial wipe which is free of any chemical that promotes the development of resistant microbial strains.

The anti-microbial effects of metallic ions such as Ag, Au, Pt, Pd, Ir (i.e. the noble metals), Cu, Sn, Sb, Bi and Zn are known. Of the metallic ions with anti-microbial properties, silver is perhaps the best known due to its unusually good bioactivity at low concentrations. This phenomenon is termed oligodynamic action. The effectiveness of silver ions notwithstanding, there remains a challenge of incorporating silver in a fabric for a wipe so that effective concentrations of silver ions are released without significantly degrading the effectiveness of the wipe over multiple uses. For example, if silver were merely incorporated in a fabric in the form of continuous metallic threads, the release of silver ions would be too slow to provide effective antimicrobial action.

What is ideally required is a wipe having a metal source capable not only of a sustained release of metal ions, but also of releasing the ions at a high enough rate to provide an effective antimicrobial wipe useful for multiple uses. One such source could be a metal coated fabric.

### SUMMARY OF THE INVENTION

The invention is a flexible antimicrobial wipe, having outer layers of a textile fabric with metallic silver coated on its individual yarns and a hydrophilic inner layer with micro fiber absorbing structure. In this disclosure, the terms “antimicrobial” and “antibacterial” are understood to be interchangeable. Dispersal of the silver on individual yarns of the fabric creates a large SSA (specific surface area, i.e., surface/volume ratio) enabling silver ions to be released more rapidly than would otherwise occur. Although various methods are available for depositing silver onto the individual yarns, an electroless or autocatalytic process is preferred.

In electroless deposition, the silver is selectively deposited on the yarn surfaces by using a suitable reducing agent to reduce the silver ions in solution to metallic silver. The silver is deposited onto any part of a yarn surface to which the solution has access.

### BRIEF DESCRIPTION OF THE DRAWINGS

FIG. 1 shows a portion of an antibacterial wipe.

FIG. 2 shows a portion of a woven outer layer of the antibacterial wipe, the layer comprising silver coated moniflament yarns.

FIG. 3 shows a portion of the woven outer layer, the layer comprising threads of made up of multiple silver-coated yarns.

FIG. 4 is a schematic of a line-of-sight deposition process.

FIG. 5 is a schematic of a plating bath for electroless deposition of silver onto a textile fabric.

### DETAILED DESCRIPTION OF THE INVENTION

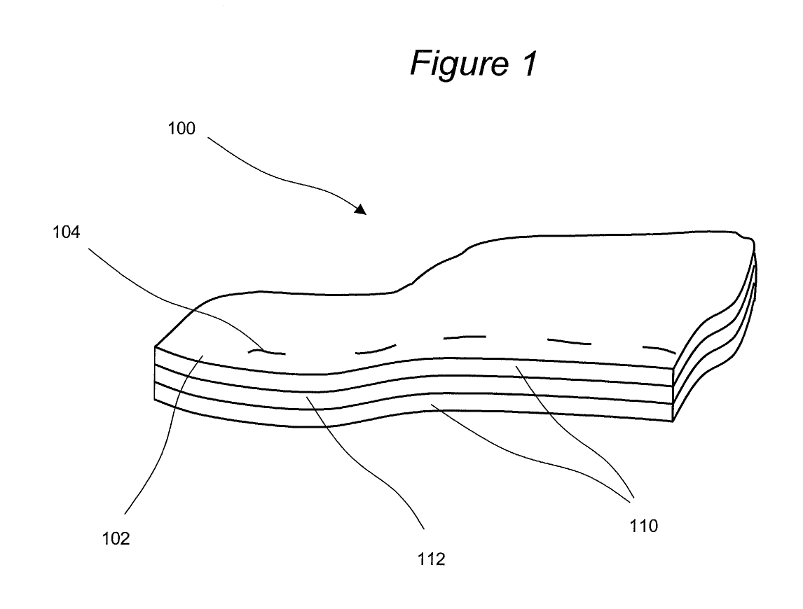

A flexible antimicrobial wipe 100 has an outer layer 110 bonded to an inner layer 112. The outer layer 110 is coated with an oligodynamic agent. The inner layer 112 is a hydrophilic material with a water retaining structure. Usually there are two opposed outer layers 110 forming a sandwich with the inner layer 112, as indicated in FIG. 1.

The outer layer 110 is preferably a woven textile fabric 120, although it can be knit, nonwoven or flock. The material of the inner layer 112 is not only readily wet by aqueous media, but also has a structure which can significantly absorb water. The inner layer 112 can have any structure that lends itself to absorbing water. Given a wettable or hydrophilic material, open cells or pores in the material can assist the absorption of water; the retention of water is also encouraged by the presence of multiple threads, between which water is drawn by capillary action. Thus, the material of the inner layer can be woven or knit, or it can be nonwoven such as a sponge material. Preferably, the inner layer 112 is made from a micro fiber material.

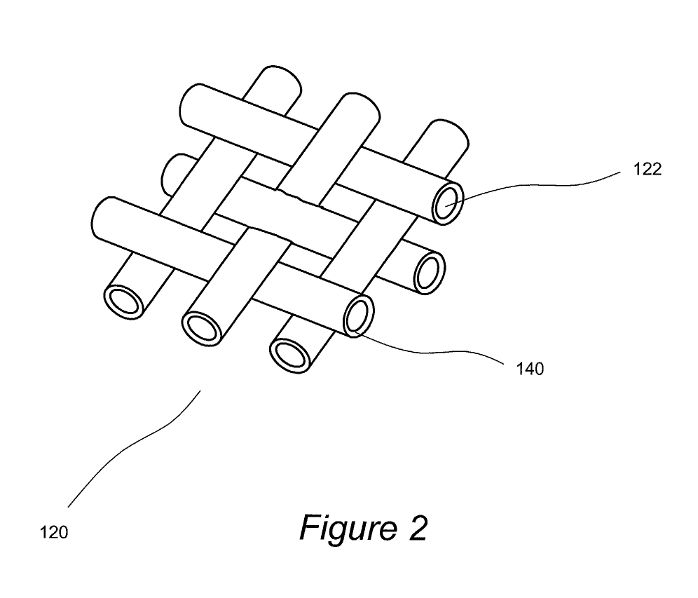

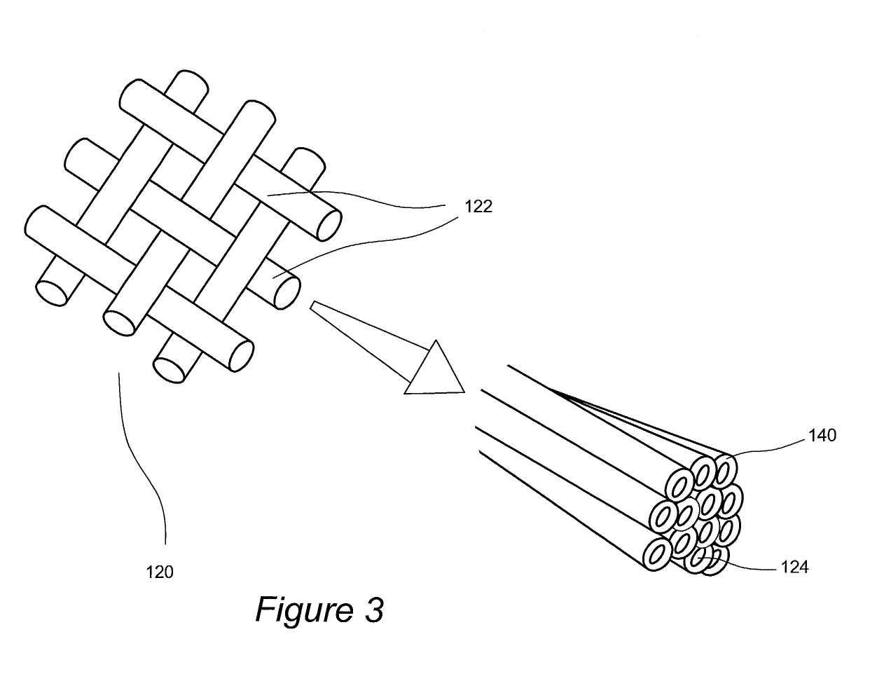

In a first embodiment, the fabric 120 is woven from threads 122 which are monofilament yarns, as shown schematically in FIG. 2. The first embodiment is currently preferred for economic reasons, since it shares the same fabric 120 with other established applications. In a second embodiment the threads 122 could be formed by spinning together multiple yarns or filaments 124, as shown in FIG. 3. The threads 122 are a substrate on which is deposited the oligodynamic agent, preferably metallic silver. Because of their small diameter, the threads 122 have a large SSA and provide a correspondingly large surface area for the deposited silver. When the threads 122 are formed from multiple yarns 124, the SSA is even greater.

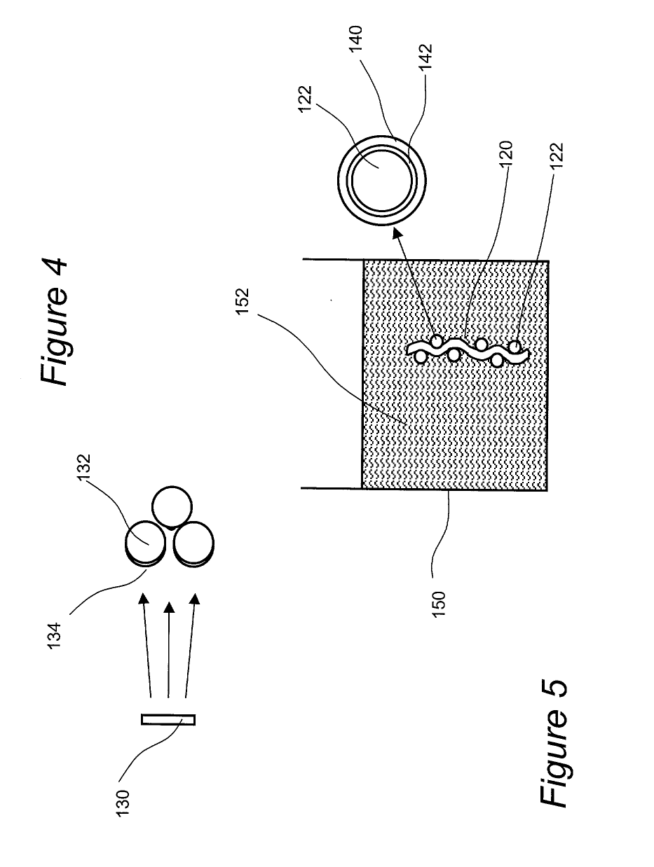

The textile fabric 120 could be coated by any of several methods for depositing a metallic coating such as silver on a substrate, including chemical plating, plasma vapor deposition (PVD), chemical vapor deposition (CVD) and combustion chemical vapor deposition (CCVD). Vapor deposition techniques are not preferred are since they rely on transferring a material in a directional manner from a deposition source 130 to a substrate 132. As is shown schematically in FIG. 4, a coating 134 is effectively deposited only on those parts of the substrate 132 in line of sight of the deposition source 130.

Some chemical plating processes which may be envisaged are not best suited for coating the textile fabric 120. Immersion or displacement plating proceeds by the reduction of a metal salt solution by electrons furnished by the substrate. This requires a metallic substrate, and furthermore deposition ceases as soon as the substrate is completely covered by the coating, since the source of electrons is no longer available. Homogeneous chemical reduction processes such as silvering rely on the reaction of a metal salt solution with a reducing agent present in the solution. However, deposition occurs indiscriminately over all objects in contact with the solution, and often in the body of the solution itself.

Electroplating requires an electrically conductive substrate, which renders it impractical for the present invention. The preferred method of producing a silver coating 140 on the textile fabric 120 of the outer layer 110 is electroless plating, also known as autocatalytic plating. This may be defined as the deposition of a metallic coating by a controlled chemical reduction that is catalyzed by the metal or alloy being deposited. Electroless plating resembles electroplating in that the process may be run continuously to build up a thick metal coating with no limit to the thickness of deposits obtainable. The electroless process deposits metal only on a catalytic surface instead of indiscriminately over all objects immersed in a plating bath. Electroless plating is thus a controlled autocatalytic chemical reduction process for depositing metals.

The source of silver for the deposition onto the textile fabric is an aqueous solution of a silver salt, such as silver nitrate. Silver ions are reduced to silver according to the reaction:

Ag⁺+e⁻=Ag°

To supply the electron for the above reaction, the solution must also contain a suitable reducing agent which may be selected from hypophosphites, borohydrides, amines, boranes, formaldehyde, hydrazine, and derivatives of these compounds. Silver is the preferred oligodynamic agent for the present invention, but other metals may be used including Au, Pt, Pd, Ir, Cu, Sn, Sb, Bi and Zn.

A great advantage of electroless plating for the present invention is that the oligodynamic metal can be deposited on any catalytic surface to which the solution has free access, with no excessive buildup on projections or edges. In particular, it can effectively penetrate between all the individual yarns of the textile fabric 120.

Briefly summarized, then, electroless deposition of silver requires a soluble silver salt providing Ag⁺ ions in aqueous solution, a reducing agent in solution, and a catalytically active surface onto which the silver is deposited. Once deposition has begun, the deposited silver itself acts as the catalyst, but if the underlying substrate is catalytically inactive, it must be so rendered by a suitable pretreatment. In general, non-metallic substrates such as many fabrics and yarns are not catalytically active.

To prepare the textile fabric 120 for coating, it is first necessary to remove sizing agents, lubricates, fingerprints and other minor soils. This is typically accomplished by mild alkaline cleaning, followed by an acid dip to remove alkaline residues.

If the material of the textile fabric 120 is not wettable by aqueous media, it must be suitably conditioned. This is accomplished by exposing it to an organic solvent or, preferably, to a plasma treatment. A resulting chemical action on the polymer surface renders it hydrophilic without causing any gross degradation of the chemical, physical, and mechanical properties of the textile. Some swelling of the textile usually accompanies this treatment.

As has been indicated, the preferred deposition process is autocatalytic, i.e., it can maintain itself once initiated. However, prior to any coating being deposited, the threads 122 of the textile fabric are usually not catalytically active relative to the desired reduction of silver ions. A sensitizing layer 142 is therefore provided on all exposed areas of the threads 122.

The preferred sensitizer is stannous chloride, although titanium (III) chloride has been used. The stannous chloride solution may typically consist of 20 g/L SnCl2 plus 40 mL/L hydrochloric acid. The solution is used at 20-25° C., and immersion time is 1 to 3 min. The free acid concentration of the sensitizing bath must be maintained by periodic additions of acid to prevent hydrolysis of the tin salt. After completion of the sensitizing step, the textile is rinsed thoroughly so that only Tin (II) ions adsorbed by the textile remain, all unadsorbed Tin (II) ions being removed.

An electroless plating bath 150 typically contains an aqueous solution 152 of silver nitrate and a reducing agent such as a hypophosphite or a borohydride. When the sensitized fabric is exposed to the plating solution, the reducing agent converts the adsorbed tin (II) ions to metallic tin, which is catalytically active. This initiates the reduction of Ag⁺ ions to metallic silver at the surface. The reduction being autocatalytic, this can continue at the surface indefinitely. Plating is continued until a coverage of 3-25% by weight of active silver is achieved. The coated fabric is then washed to remove excess chemical and dried. The steps of electroless coating can be performed as a batch or continuous process.

Preferably, the finished wipe 100 is made by bonding two outer layers 110 of the silver-coated textile fabric 120 and the hydrophilic inner layer 112. However, it is also possible for the wipe 100 to have only a single outer layer 110. Bonding can be by thermal welding, ultrasonic welding, sewing, or by a suitable adhesive. Since the system performs best when the layers are not in intimate contact in the working areas, the layers are typically bonded only around the perimeter of the wipe 100, for example by stitches 104 as shown in FIG. 1. Optionally, the layers may be bonded together at locations within the perimeter, for example by spot welding at different points.

Nylon is typically used as the silver-bearing textile fabric for the outer layers. However, some forms of nylon have little affinity for cationic plating such as occurs in electroless deposition. Polymerization reactions known in the art for making nylon can be modified in various ways to achieve such affinity. For example, N-aminoethylpiperazine or sulphonic acid groups can be introduced. One modification that provides affinity for cationic plating is obtained by adding a certain amount of sulphoisophthalic acid prior to polymerization.

A wide variety textile fabric weights can be used, typically in the range 0.5 to 8 oz per square yard. A woven fabric can have any weight and thread count consistent with flexiblity and an adequate capacity for holding silver. Warp and weft threads in a woven fabric can either be monofilaments or they can be spun from multiple individual yarns. Such factors are determined by the need to balance factors such as strength, flexibility and SSA. The weight of the micro fiber absorbing structure of the inner layer can be in the range 0.3 to 50 oz per square yard.

This wipe 100 of this invention can be used multiple times without significant loss of its antimicrobial properties. Washing can include machine washing in cold water with normal detergents, or hand washing and air-drying. Since it does not depend on volatile constituents, the wipe 100 of this invention has a long shelf life and can be stored for indefinite periods of time at temperatures of −100° C. to +100° C. with no deterioration of its anti-microbial properties. By contrast, conventional anti-bacterial chemical based products need special packaging to retain their volatile antibacterial agents.

Assays of Oligodynamic Activity

A. Time Kill Study

1. Sample was divided into 1″×1″ square swatches. Similar swatches were cut from a non silver bearing control fabric. Sample and control swatches were placed in sterile petri dishes.

2. Swatches were inoculated with 100 μL each of a 1,000,000 cfu/mL test strain.

3. One swatch of silver plated fabric and of control fabric was removed after 30 minutes, 60 minutes & 180 minutes and placed in 9 mL of Letheen broth.

4. Letheeen broth/swatch solutions were vortexed and serially plated to Tryptic Soy Agar. The dishes were incubated at 30-35° C. for 2-3 days.

5. Control fabric counts (C) and silver plated fabric counts (S) were compared and a percent reduction calculated (Table 1), as follows:

Percent reduction for test piece at time t relative to control at time t:

P(C)ₜ₌100×(Cₜ−Sₜ)/Cₜ

Percent reduction for test piece at time t relative to test piece at time zero:

P(0)ₜ=100×(S₀−Sₜ)/Cₜ

TABLE 1
time kill data for the test strain Psuedomonas aeruginso ATCC9027

| Time, t (minutes) | 30 | 60 | 180 |
|---|---|---|---|
| Test and control pieces at time 0 | 111,000 | 111,000 | 111,000 |
| Control piece at time t | 111,000 | 108,000 | 106,000 |
| Test piece at time t | 63,000 | 5,000 | 40 |
| P(c)ₜ | 43.243 | 95.370 | 99.962 |
| P(0)ₜ | 43.243 | 95.495 | 99.964 |

The data of Table 1 confirm that the silver plated fabric has far greater oligodynamic activity than the control fabric.

B. Kirby-Bauer Standard Antimicrobial Susceptibility Test

Swatches of the test fabric 20 mm square were incubated in vessels containing agar containing selected test organisms. Inhibition of organism growth around a given swatch was determined as a width W in mm of a zone of inhibition extending outward from the piece; the greater W, the greater the inhibition. Growth inhibition beneath the swatches was also determined.

TABLE 2
Kirby-Bauer test data

| | | | Inhibition |
|---|---|---|---|
| | Incubation | W | beneath |
| Test organism | regime | mm | swatch |
| Staphylococcus aureus | 1 | 3 | yes |
| ATCC 33591 | | | |
| Psuedomonas aeruginosa | 1 | 2 | yes |
| ATCC 9027 | | | |
| Entereocoocus faecalis | 1 | 1 | yes |
| ATCC 51575 | | | |
| Candida albicans | 2 | 0 | yes |
| ATCC 10231 | | | |

Incubation regimes 1: 35-37° C. for 16-24 hours; 2: 30° C. for 48 hours

The data of Table 2 indicate some degree of oligodynamic activity for all the selected test organisms with respect to the silver-coated test swatches.

C. Comparative Zone of Inhibition Study

This test (Table 3) is similar to the Kirby-Bauer test, but with different incubation regimes and using a cotton control fabric. The culture media were tryptic soy agar (TSA) for bacteria and potato dextrose agar (PDA) for yeast and mold.

TABLE 3
Comparative inhibition data

| | | | | | Silver-coated | |
|---|---|---|---|---|---|---|
| | | | Control piece | | test piece | |
| | | | | Inhib. | | Inhib. |
| | Incubation | Culture | W | beneath | W | beneath |
| Test organism | regime | medium | mm | swatch? | mm | swatch? |
| P. aeruginosa | 3 | TSA | 0 | no | 17.1 | yes |
| ATCC 9027 | | | | | | |
| S. aureus | 3 | TSA | 0 | no | 0 | yes |
| ATCC 6538 | | | | | | |
| C. albicans | 4 | PDA | 0 | no | 0 | yes |
| ATCC 10231 | | | | | | |
| A. Niger | 4 | PDA | 0 | no | 0 | yes |
| ATCC 16404 | | | | | | |

Incubation regimes 3: 30-35° C. for 2 days; 4: 20-25° C. for 4 days

The comparative zone of inhibition data of Table 3 confirm the activity of the silver-coated material.

The invention can be used as an anti-bacterial kitchen wipe for sanitizing the food preparation areas while cleaning up general food debris for these areas. It can also be used as a anti-microbial wipe for public and private food eating areas such as restaurants and public eateries, where tables need to be kept clean and free of microbial contamination. It can further be used in hair and nail salons to manage the propagation of bacteria and fungi at the workstations, foot bathes and sinks.

Although solid metal fibers of silver can be woven into a cloth, a wipe made from such a cloth is much more expensive than the present invention, which is as economical as conventional alternatives. Since nylon or other conventional threads are typically thinner than solid metal fibers, the SSA is correspondingly greater and allows more rapid release of silver ions. The SSA is even greater when the textile threads are composed of multiple yarns. Further, the ability to efficiently deposit silver on all surfaces of an existing textile allows great versatility as to what textiles can be used, as compared to having to pre-coat threads before weaving.

The selectivity of electroless deposition of metallic silver renders it more economical than other processes in which metallic silver may be formed at undesired sites. Electroless deposition results in finely deposited crystals that further enhance the release of silver ions to the area being wiped.

The economy of depositing silver over solid metal fibers makes the product cost-competitive with chemical alternatives. Further the technology to deposit silver on woven fabric vs. yarn that would have to be woven gives the product great versatility in what base materials can be used for best results in the application.

Various features of the present invention have been described with reference to the above embodiments. It should be understood that modification may be made without departing from the spirit and scope of the invention as represented by the following claims.

What is claimed:

1. An anti-microbial wipe, comprising:
(a) a silver impregnated flexible textile layer; and
(b) a fibrous water-retaining layer connected to the textile layer.

2. The anti-microbial wipe of claim 1, wherein the textile layer is formed of a plurality of yarns, each yarn including silver.

3. The anti-microbial wipe of claim 1, further comprising a second silver impregnated flexible textile layer connected to one of the textile layer and the water retaining layer to dispose the water retaining layer intermediate the flexible textile layer and the second silver impregnated flexible textile layer.

4. The anti-microbial wipe of claim 1, wherein the textile layer is between 3% and 25% by weight active silver.

5. The anti-microbial wipe of claim 1, wherein the water retaining layer is bonded to a portion of the flexible textile layer.

6. The anti-microbial wipe of claim 1, wherein the water retaining layer is a micro fiber structure having a weight of approximately 0.3 to 50 oz. per square yard.

7. The anti-microbial wipe of claim 1, wherein the fibrous water retaining layer is hydrophilic.

8. The anti-microbial wipe of claim 2, wherein the silver is deposited on the yarns by a plating process.

9. The anti-microbial wipe of claim 2, wherein the plating process is one of PVD, CVD, CCVD and electroless coating.

10. The antimicrobial wipe of claim 1, wherein the silver impregnated flexible textile layer is a woven fabric with warp threads and weft threads.

11. The antimicrobial wipe of claim 10, wherein each of the warp threads and weft threads comprises a plurality of yarns.

12. The antimicrobial wipe of claim 1, wherein the flexible textile layer has a weight of approximately 0.5 to 8 oz. per square yard.

13. An anti-microbial wipe, comprising:
(a) a textile cationic impregnated fabric layer; and
(b) a fibrous water retaining layer connected to the fabric layer.

14. The anti-microbial wipe of claim 13, wherein the water retaining layer is a micro fiber structure having a weight of approximately 0.3 to 50 oz. per square yard.

15. The anti-microbial wipe of claim 13, wherein the fibrous water retaining layer is hydrophilic.

16. The anti-microbial wipe of claim 13, wherein the cationic impregnated fabric layer includes silver.

17. The anti-microbial wipe of claim 16, wherein the silver is deposited on the yarn by physical vapor deposition, chemical vapor deposition including electroless deposition or plating.

18. The anti-microbial wipe of claim 13, wherein the fabric layer is approximately 3 percent to 18 percent weight of silver.

19. An anti-microbial wipe, comprising:
(a) a textile fabric impregnated with an oligodynamic anti-microbial agent; and
(b) a micro fiber hydrophilic moisture retaining layer connected to the textile layer.

20. The anti-microbial wipe of claim 19, wherein the fabric has approximately 3 to 18 percent weight of silver.

21. The anti-microbial wipe of claim 19, wherein the anti-microbial wipe is free of volatile anti-microbial agents.

22. The anti-microbial wipe of claim 19, wherein the oligodynamic anti-microbial agent is selected from the group of elements consisting of Ag, Au, Pt, Pd, Ir, Cu, Sn, Sb, Bi and Zn.

23. The anti-microbial wipe of claim 19, wherein the textile fabric is a nylon composition.

24. The anti-microbial wipe of claim 23, wherein the nylon composition has been modified to provide an affinity for cationic plating.
